(19) United States
(12) Patent Application Publication
(10) Pub. No.: US 2013/0040405 A1
(43) Pub. Date: Feb. 14, 2013

(54) METHODS FOR SENSITIVE AND RAPID DETECTION OF MOLECULES

(71) Applicants: Laura Segatori, Houston, TX (US); Sibani Lisa Biswal, Houston, TX (US); Jinghui Wang, Houston, TX (US); Patricia D. Palena, Plainsboro, NJ (US); Michael J. DiMario, Skillman, NJ (US)

(72) Inventors: Laura Segatori, Houston, TX (US); Sibani Lisa Biswal, Houston, TX (US); Jinghui Wang, Houston, TX (US); Patricia D. Palena, Plainsboro, NJ (US); Michael J. DiMario, Skillman, NJ (US)

(73) Assignees: Lockheed Martin Corporation, Moorestown, NJ (US); William Marsh Rice University, Houston, TX (US)

(21) Appl. No.: 13/584,419

(22) Filed: Aug. 13, 2012

Related U.S. Application Data
(60) Provisional application No. 61/522,535, filed on Aug. 11, 2011.

(51) Int. Cl. G01N 33/557; G01N 29/036; G01N 21/21; G01N 21/55

(52) U.S. Cl. 436/501

(57) ABSTRACT

In some embodiments, the present invention provides methods of detecting a molecule in a sample, such as an explosive. In some embodiments, the method comprises: associating the sample with an antigen/binding agent complex; measuring a rate of displacement of the binding agent from the antigen by the molecule in the sample; and correlating the measured rate of displacement to the presence of the molecule in the sample. In some embodiments, the measuring step comprises a determination of a change in frequency of the sample and a change in energy dissipation of the sample over a time interval. In some embodiments, the correlating step comprises a calculation of a ratio of a change in energy dissipation of the sample over a change in frequency of the sample over the time interval. In some embodiments, the method is used to determine the molecule concentration in the sample.

### CROSS-REFERENCE TO RELATED APPLICATIONS

This application claims priority to U.S. Provisional Patent Application No. 61/522,535, filed on Aug. 11, 2011. The entirety of the aforementioned application is incorporated herein by reference.

### BACKGROUND

Currently available sensors and methods for detecting various molecules (e.g., explosives, such as TNT) suffer from various limitations. Such limitations include low specificity, low sensitivity and prolonged detection periods. These limitations may in turn interfere with the rapid, sensitive and specific detection of molecules of interest from crude environments. Therefore, a need exists for the development of improved methods and sensors for molecular detection.

### BRIEF SUMMARY

In some embodiments, the present disclosure provides methods of detecting molecules in a sample. In some embodiments, such methods comprise: associating the sample with an antigen/binding agent complex; measuring a rate of displacement of the binding agent from the antigen by the molecule in the sample; and correlating the measured rate of displacement to the presence of the molecule in the sample. In some embodiments, the measuring step may further comprise a determination of a change in frequency of the sample, and a change in energy dissipation of the sample over a time interval. In further embodiments, the correlating step may also comprise a calculation of a ratio of a change in energy dissipation of the sample over the change in frequency of the sample over the time interval. In some embodiments, the correlating step may also comprise calculating the slope of a plot, where the plot reflects the change in energy dissipation of the sample over the change in frequency of the sample over the time interval.

In some embodiments, the methods are utilized to detect from a sample a molecule of interest, such as an explosive (e.g., TNT). In some embodiments, the methods are utilized to determine the concentration of the molecule in the sample.

In some embodiments, the methods may utilize at least one of a quartz crystal microbalance (QCM), surface plasmon resonance (SPR), ellipsometry, microcantilevers, optical microcavities, or Langmuir kinetic models. In some embodiments, the sample may be in a liquid state, a gaseous state, a solid state, or a combination of such states. In some embodiments, the sample may be derived from a gaseous environment. In some embodiments, the sample may be derived from a liquid environment. In some embodiments, the sample may be derived from a crude environment, such as seawater.

In some embodiments, the molecule to be detected may be an explosive. In some embodiments, the explosive to be detected may include at least one of nitroglycerin, 2,4,6-trinitrotoluene (TNT), pentaerythritol tetranitrate (PETN), octahydro-1,3,5,7-tetranitro-1,3,5,7-tetrazocine (HMX), nitrocellulose, analogs thereof, derivatives thereof, and combinations thereof.

In some embodiments, the molecule to be detected is TNT. In some embodiments, the TNT to be detected may be at least one of 2,4,6-trinitrotoluene, analogs thereof, derivatives thereof, and combinations thereof. In some embodiments, the TNT to be detected is 2,4,6-trinitrotoluene.

In some embodiments, the antigen/binding agent complex may be immobilized on a surface of a plate, such as a quartz crystal plate. In some embodiments where the molecule to be detected is TNT, the antigen may be at least one of 2,4,6-trinitrotoluene, analogs thereof, derivatives thereof, and combinations thereof. In further embodiments, the antigen may be at least one of 1,3,5-Trinitrobenzene (TNB), 2,6-Dinitrotoluene (DNT), 2-Amino-4,6-Dinitrotoluene (2-a-DNT), 2,4,6-trinitrobenzene (TNB), 2,4,6-trinitrobenzene sulfonic acid (TNBS), derivatives thereof, and combinations thereof.

In some embodiments, the binding agent has an affinity for the antigen, and an affinity for the molecule. In some embodiments, the binding agent may be at least one of aptamers, peptides, peptide nucleic acids, antibodies, proteins, RNA, DNA, small molecules, dendrimers, and combinations thereof. In some embodiments where the molecule to be detected is TNT, the binding agent may be an anti-TNT antibody, such as a monoclonal antibody. In some embodiments, the binding agent is unlabeled.

Additional embodiments of the present disclosure pertain to sensors for detecting molecules in accordance with the aforementioned methods. Such sensors may include the antigen/binding agent complexes immobilized onto a surface, such as a quartz crystal plate.

The methods and sensors of the present disclosure provide numerous advantages. For instance, the methods and sensors of the present disclosure may be utilized to specifically and rapidly detect trace levels of explosives (e.g., TNT) from crude environments. In some embodiments, the methods and sensors of the present disclosure may be used to detect nanogram levels of explosives (e.g., TNT) in a matter of seconds or minutes from crude environments that contain other similar chemicals.

### BRIEF DESCRIPTION OF THE FIGURES

FIG. 1 illustrates a method of detecting molecules in a sample.

FIG. 2 illustrates the displacement of anti-2,4,6-trinitrotoluene (TNT) antibodies by TNT and TNT analogs. FIG. 2A shows enzyme-linked immunosorbent assay (ELISA) measurements of anti-TNT antibody displacement from 1,3,5-Trinitrobenzene (TNB) by TNT and TNT analogs. Compounds were tested at concentrations ranging from 0.1 ng/mL to 100 μg/mL. The anti-TNT antibody (A1.1.1) concentration was at 10 μg/mL. Data points were obtained from at least three independent experiments and normalized to the signal measured from control samples containing only antibody. The detection limit for each molecule corresponds to the lowest concentration that causes loss of absorbance. FIG. 2B shows the chemical structure of TNT and TNT analogs used in this study.

FIG. 3 shows quartz crystal microbalance (QCM) measurements for the detection of TNT. The measurements were performed during anti-TNT antibody displacement from TNB by TNT and TNT analogs. Anti-TNT antibody (10 μg/mL) was immobilized on the crystal surface and three compounds (10 μg/mL, TNT: blue line; TNB: red line; DNT: brown line) were tested. The arrows represent the time points when TNT and its analogs were added. Results are representative of three independent experiments.

FIG. 4 shows plots representing dissipation energy change (ΔD) as a function of frequency change (Δf) in samples undergoing anti-TNT antibody displacement by TNT and TNB, as measured by QCM. FIG. 4A shows ΔD-ΔF plots of samples that were incubated with TNT at 1 μg/mL. FIG. 4B shows ΔD-ΔF plots of samples that were incubated with TNB at 10 μg/mL. Colored points represent different overtones of frequency in QCM measurement. The slopes are obtained from the trend lines of these points. The difference in slopes distinguishes TNT from its analogs.

FIG. 5 shows data relating to the calculation of TNT concentrations from various samples. FIG. 5A illustrates TNT detection by the QCM based TNT sensor. A solution of TNT at concentrations ranging from 0.1 ng/mL to 10 μg/mL was flowed on to the QCM crystal, and the normalized frequency change was calculated. The data points reported were obtained at equilibrium from at least three independent experiments. FIG. 5B shows a “quick” slope analysis for rapid detection of TNT. The concentration of TNT in the solutions analyzed ranged from 0.1 ng/mL to 10 μg/mL, and the time interval chosen was 10 minutes. The data points reported were obtained from at least three independent experiments.

FIG. 6 shows data relating to the detection of TNT in crude environments. The limit of detection of TNT in PBS (green), fertilizer (blue), and seawater (red) was 0.1 ng/mL. The black line represents the value of control sample (solutions without TNT). The data analysis was conducted as described in the Examples (Equation 2). Data points were obtained from at least three independent experiments.

FIG. 7 shows a calculation of competitive binding affinity K_{A}—. [TNT]/ΔF is plotted with respect to [TNT], according to Equation 4 (see Examples). Four data points were used to maximize data fitting. R-squared value for this plot is 0.9977. From this linear relationship, the slope and intercept of this plot can be obtained. Maximal frequency change (ΔFₘₐₓ) and competitive binding affinity (K_{A}) were calculated.

FIG. 8 shows a calculation of positive reaction rate K_{A}. In Equation S-5 (see Examples), the right part is plotted as a function of time. R-squared value for this plot is 0.9983. The slope represents the value of the positive reaction rate (K_{A}).

FIG. 9 shows a mathematical model of TNT (10 μg/mL) detection. The solid line represents the simulated data obtained from Equation 5 (see Examples), while the open circles are experimental data points obtained from QCM.

FIG. 10 shows a mathematical model of TNB (10 μg/mL) detection. The solid line represents the simulated data obtained, while the open squares are experimental data points obtained from QCM.

FIG. 11 shows a mathematical model of DNT (10 μg/mL) detection. The solid line represents the simulated data obtained, while the open triangles are experimental data points obtained from QCM.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The rapid and reliable detection of explosives has gained increasing attention, due to health and public safety reasons. Most types of currently used explosives are toxic to living systems, even when present in trace amounts. For instance, a variety of technologies have been developed for the detection of 2,4,6-trinitrotoluene (TNT). They can be broadly classified as physical, chemical and biological methods, based on the detection mechanism and output signal. Physical methods, such as laser, mass spectroscopy and NMR, allow achieving high sensitivity of detection, but involve time consuming and costly procedures. Particularly, TNT concentrations as low as 1 pg/mL can be detected in vapor phase with low false positive signals by using laser photoacoustic spectroscopy.

The specificity of chemical methods, including electrochemical sensors and fluorescence spectrophotometry, is relatively low. Fluorescence spectrophotometry is based on the electron deficiency of TNT. As with many nitroaromatic compounds, TNT functions as an electron acceptor and causes quenching of a number of photoluminescent, fluorescent, and phosphorescent materials by electron transfer. Among these materials, fluorescent polymers were first reported to allow detecting saturated TNT vapor (0.1 ng/mL) within seconds. The performance of fluorescence spectrophotometry was later improved using mesostructured silica films, nanocrystals, and quantum dots. Particularly, the limit of detection for TNT was reduced to ˜0.023 ng/mL using a hybrid material composed of gold nanorod and quantum dots. This method, however, exhibits relatively low specificity, which prevents distinguishing TNT from other nitroaromatic compounds with similar chemical properties.

Biological methods typically present enhanced specificity due to the use of TNT specific molecules, such as antibodies, and molecularly imprinted polymers (MIPs). Most of these reported sensors lack data comparing their performance in a crude environment. Furthermore, one of the critical issues in the development of a TNT sensor is the small size of the TNT molecule that often precludes high sensitivity of detection at low concentrations. Therefore a need exists for the development of rapid and accurate sensors that combine the high sensitivity of chemical methods with the high specificity of biological methods for the detection of various explosives (e.g., TNT) and other molecules of interest in aqueous solutions that contain similar molecules. The present disclosure addresses this need.

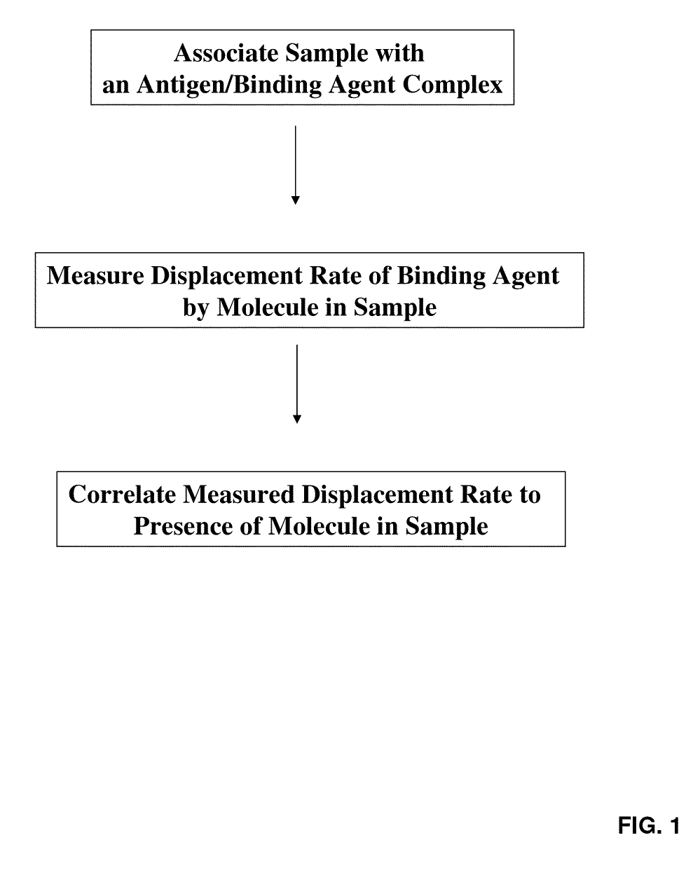

In some embodiments, the present disclosure pertains to methods for detecting molecules (e.g., TNT) in samples derived from various environments. As illustrated in FIG. 1, such methods generally include: (1) associating the sample with an antigen/binding agent complex; (2) measuring a rate of displacement of the binding agent from the antigen by the molecule in the sample; and (3) correlating the measured rate of displacement to the presence of the molecule in the sample. In some embodiments, the methods are utilized to detect the concentration of the molecule in the sample. In some embodiments, the methods are utilized to detect nanogram levels of the molecule in the sample.

Additional embodiments of the present disclosure pertain to sensors for detecting molecules in various samples. As set forth in more detail herein, the methods and sensors of the present disclosure have numerous embodiments and variations.

Molecules

As set forth herein, the methods of the present disclosure may be utilized to detect and quantify various molecules from various samples. In some embodiments, the molecule to be detected may include an explosive. In some embodiments, the explosive may be at least one of nitroglycerin, 2,4,6-trinitrotoluene (TNT), pentaerythritol tetranitrate (PETN), octahydro-1,3,5,7-tetranitro-1,3,5,7-tetrazocine (HMX), nitrocellulose, analogs thereof, derivatives thereof, and combinations thereof.

In some embodiments, the molecule to be detected may be TNT. In various embodiments, the TNT to be detected and analyzed in the samples may include 2,4,6-trinitrotoluene, analogs thereof, derivatives thereof, and combinations thereof.

Samples

In some embodiments, the samples may be derived from a gaseous environment, a liquid environment, a solid environment, or combinations of such environments. In some embodiments, the sample may be derived from a crude environment, such as from seawater, fertilizers, wastewater, sludge, air, and other similar environments.

In various embodiments, the samples to be analyzed may contain various types of chemicals. In some embodiments, the samples may contain non-modified form of TNT (i.e., 2,4,6-trinitrotoluene). In some embodiments, the samples may contain analogs or derivatives of TNT, as previously described. In some embodiments, the samples to be analyzed may contain molecules with similar chemical properties and structures as TNT.

The samples to be analyzed may also be in various states. In some embodiments, the sample may be in a liquid state. In some embodiments, the sample may in a gaseous state. In some embodiments, the sample may be in a solid state. In some embodiments, the sample to be analyzed may be in a gaseous state and a solid state.

Antigen/Binding Agent Complexes

In various embodiments, obtained samples may be exposed to an antigen/binding agent complex. As set forth herein, various antigens and binding agents may be utilized for such purposes.

Antigens

Antigens generally refer to molecules, surfaces or objects that are capable of binding to a binding agent. In some embodiments, antigens compete with the molecule that is to be detected in a sample. For instance, in some embodiments that are used for TNT detection, the antigen may compete with TNT or TNT analogs for binding to a TNT binding agent. In further embodiments that are used for TNT detection, the antigens may include, without limitation, 2,4,6-trinitrotoluene (TNT), analogs thereof, derivatives thereof, and combinations thereof. In additional embodiments, the antigens may include at least one of 1,3,5-Trinitrobenzene (TNB), 2,6-Dinitrotoluene (DNT), 2-Amino-4,6-Dinitrotoluene (2-a-DNT), 2,4,6-trinitrobenzene (TNB), 2,4,6-trinitrobenzene sulfonic acid (TNBS), derivatives thereof, and combinations thereof. In some embodiments, the antigen may be a TNB-ovalbumin complex. In some embodiments, the antigen and the molecule to be detected may be the same compound.

In some embodiments, the antigen may be immobilized onto a surface of an object, such as a plate (e.g., quartz crystal plate). In some embodiments, the antigen may be immobilized onto a surface by covalent or non-covalent associations with the surface. In some embodiments, the antigen may be an actual surface of an object, such as a quart crystal plate. In some embodiments, the antigen is a TNB-ovalbumin complex immobilized onto a surface of a quartz crystal plate.

Binding Agents

Binding agents generally refer to molecules that are capable of binding to an antigen and a molecule to be detected from a sample. In some embodiments, the binding agent has an affinity for the antigen, and an affinity for the molecule. In some embodiments, the affinity of the binding agent for the molecule may be equal to or substantially equal to the affinity of the binding agent for the antigen. In some embodiments, the affinity of the binding agent for the molecule may be higher than the affinity of the binding agent for the antigen. In some embodiments, the affinity of the binding agent for the molecule may be lower than the affinity of the binding agent for the antigen.

In some embodiments, the binding agent may include at least one of aptamers, peptides, peptide nucleic acids, antibodies, proteins, RNA, DNA, small molecules, dendrimers, and combinations thereof. In some embodiments that are used for TNT detection, the binding agent may include an anti-TNT antibody. In some embodiments, the anti-TNT antibody may be a monoclonal antibody. In some embodiments, the anti-TNT antibody may include at least one of monoclonal antibodies, polyclonal antibodies, antibody fragments, and combinations thereof.

In some embodiments, the binding agents may be linked to a marker. In some embodiments, the marker may include at least one of radioactive markers, fluorescent markers, enzyme-based markers, and combinations thereof. In some embodiments, the marker may be horseradish peroxidase (HRP). In some embodiments, the binding agents may be unlabeled (i.e., not linked to any markers).

Binding agents may associate with antigens in various manners. In some embodiments, the association may be non-covalent. In some embodiments, the binding agents may be non-covalently associated with antigens through one or more types of interactions, such as sequestration, adsorption, ionic bonding, dipole-dipole interactions, hydrogen bonding, Van der Waals interactions, and combinations of such interactions.

In some embodiments, the binding agents may saturate the binding sites of the antigens. In some embodiments, the binding agents may bind to antigens without saturating their binding sites. In some embodiments, the antigen/binding agent complexes may reach equilibrium.

As set forth in more detail herein, the associations between binding agents and antigens may be displaced by a molecule in a sample (e.g., TNT). The displacement rate may then be measured to determine the presence of the molecule within the sample.

Measuring Rate of Displacement of Binding Agents

Various methods may be used to measure the rate of displacement of binding agents from antigens by a molecule in a sample. Such methods may include the utilization of at least one of quartz crystal microbalance (QCM), surface plasmon resonance (SPR), ellipsometry, microcantilevers, optical microcavities, a Langmuir kinetic model, and combinations thereof. In some embodiments, QCM may be utilized to measure the rate of displacement. QCM has been used as biosensors in studies of affinity estimation and polymer conformational changes due to its high sensitivity, label-free detection, real-time measurements, portability, and ease of operation. In some embodiments, a Langmuir kinetic model may also be used to measure the rate of displacement.

In some embodiments, the rate of displacement of binding agents from antigens may include the determination of a change in frequency of a sample over a time interval. In some embodiments, the change in frequency may be normalized. In some embodiments, the change in frequency may be measured in Hz. In some embodiments, the change in frequency may be determined by QCM or the Langmuir kinetic model.

In some embodiments, the rate of displacement of binding agents from antigens may include the determination of a change in energy dissipation of a sample over a time interval. In some embodiments, the rate of displacement of binding agents from antigens may include a determination of a change in frequency and a change in energy dissipation of a sample over a time interval.

Correlating Rate of Displacement to Presence of a Molecule

The measured rate of displacement of binding agents from antigens by molecules within a sample may be correlated to the presence of the molecule in the sample by various methods (hereinafter the correlating step). In some embodiments, the correlating step may be used to determine the concentration of the molecule in the sample. In some embodiments, the correlating step may include a calculation of the change in output signal of the sample containing the molecule over a time interval. In some embodiments, such methods may be referred to as the “quick slope” method. In some embodiments, the quick slope method may include a calculation of a ratio of a change in energy dissipation of the sample over the change in frequency of the sample over a time interval. In some embodiments, the “quick slope” method may include calculating the slope of a plot, where the plot reflects the change in energy dissipation of the sample over the change in frequency of the sample over the time interval.

In some embodiments, the quick slope method can allow estimating the dependence of the rate of displacement on the concentration of a molecule that is to be detected in a sample (e.g., TNT). For instance, the quick slope method can be used to accurately determine concentrations of a desired molecule (e.g., TNT) as low as 0.1 ng/ml from crude samples.

Sensors

Additional embodiments of the present disclosure pertain to sensors for detecting and quantifying a desired molecule (e.g., TNT) from a sample. In some embodiments, the sensors may be QCM-based sensors. In some embodiments, the sensors may include the aforementioned antigen/binding agent complexes on a surface. In some embodiments, the surface may be a plate, such as a quartz crystal plate. In some embodiments, the sensors may be utilized to detect or quantify a desired molecule (e.g., TNT) from various samples, and in accordance with the methods of the present disclosure.

Applications and Advantages

The methods and sensors of the present disclosure provide numerous applications and advantages. In particular, the sensors and methods of the present disclosure can provide fast, sensitive and specific methods of detecting a desired molecule (e.g., explosives, such as TNT) from various environments (e.g., aqueous environments).

For instance, unlike conventional methods and sensors that may take hours to detect the presence of explosives or other molecules, the methods and sensors of the present disclosure can be used to detect the presence of explosives (e.g., TNT) within seconds or minutes. Furthermore, in some embodiments of the present disclosure, the methods for detecting a desired molecule in a sample may take anywhere from 10 seconds to about 60 minutes. In some embodiments, the methods of the present disclosure may take about 10 minutes to detect a desired molecule (e.g., TNT) in a sample.

Likewise, the sensors and methods of the present disclosure can be capable of detecting trace levels of a desired molecule (e.g., TNT) from various environments. For instance, in some embodiments, the methods and sensors of the present disclosure may be capable of detecting nanogram levels of a desired molecule (e.g., TNT) in a sample. In some embodiments, the methods and sensors of the present disclosure may detect or be capable of detecting between about 0.1 ng/ml to about 10 μg/ml of a desired molecule (e.g., TNT) in a sample.

Without being bound by theory, it is envisioned that the sensitivity of the methods of the present disclosure can be due to signal amplification from the displacement of binding agents from antigens by a desired molecule in a sample. Because the molecular weight of the binding agent is usually greater than that of the molecule, detecting the frequency change caused by the binding agent displacement, rather than that associated with the binding of the molecule to the binding agent, gives rise to significant amplification of the detection signal.

Furthermore, the methods and sensors of the present disclosure can be capable of detecting various molecules (e.g., explosives, such as TNT) from various environments that contain molecules with similar chemical properties or structures. For instance, in some embodiments that are used for TNT detection, the rate of binding agent displacement can be proportional to the affinity of the TNT in solution for the binding agent. Thus, in some embodiments, TNT can be rapidly detected by using a binding agent with high specificity for TNT and low specificity for other molecules with similar chemical structures, or with different chemical structures but similar explosive properties.

### ADDITIONAL EMBODIMENTS

Reference will now be made to more specific embodiments of the present disclosure and experimental results that provide support for such embodiments. However, Applicants note that the disclosure below is for illustrative purposes and is not intended to limit the scope of the claimed subject matter in any way.

The Examples herein pertain to the sensitive detection of 2,4,6-trinitrotoluene (TNT) using competition assay on Quartz Crystal Microbalance. Currently available TNT sensors are characterized by high sensitivity, but low specificity, which limits the detection of TNT in crude environments. Applicants report in the Examples herein a TNT sensor designed to measure the displacement of a TNT-specific antibody by quartz crystal microbalance (QCM). This sensor combines high sensitivity of detection (e.g., 0.1 ng/mL) with the ability to distinguish TNT from molecules with similar chemical properties. Particularly, the reliability of this method for the detection of TNT in crude environments was investigated by using fertilizer solution and artificial seawater. Instead of measuring actual binding of TNT, the method described is based on the displacement of an anti-TNT antibody, which allows quantifying the concentration of TNT in solution with higher sensitivity. In addition, by utilizing the rate of antibody displacement, the detection time is significantly decreased from hours, which would be necessary to measure the frequency change at equilibrium, to minutes. A Langmuir kinetic model was used to describe the molecular interactions on the surface of the sensor and to establish a standard curve to estimate on-site TNT detection.

### Example 1

### Materials and Methods

2,4,6-Trinitrotoluene (TNT), 1,3,5-Trinitrobenzene (TNB), 2,6-Dinitrotoluene (DNT), 2-Amino-4,6-Dinitrotoluene (2-a-DNT), and ovalbumin were purchased from Sigma-Aldrich. Anti-TNT monoclonal antibody (A1.1.1) was purchased from Strategic Diagnostics. HRP conjugated anti-mouse antibody was purchased from Assay Designs. 2,4,6-trinitrobenzene sulfonic acid (TNBS) and Dithiobis[succinimidyl propionate] (DSP) were purchased from Pierce. TMB substrate was purchased from BioLegend.

### Example 2

### Synthesis of TNB-Ovalbumin Complex

The TNB-ovalbumin complex was prepared by conjugating the sulfonic group of 2,4,6-trinitrobenzene sulfonic acid (TNBS) to the primary amines of ovalbumin molecules, as previously described. J. Immunol. 1979. 123: 426-433. Briefly, a solution of 10.2 mM TNBS and 0.67 mM ovalbumin in PBS (pH 8.0) was stirred at 30 rpm for one hour at room temperature. The reaction product was dialyzed overnight against PBS to eliminate free TNBS and stored at −80° C. until use.

### Example 3

### Preparation of TNT and TNT Analogs

Acetonitrile was evaporated from the stock solution of TNT (1000 g/mL) and TNT analogs. Then, PBS buffer, fertilizer solution or seawater, was used to dissolve them before use. Commercially available fertilizer powder was dissolved in PBS buffer at a concentration of 1 mg/mL (0.1% w/v), and the pH was adjusted to 7.4. Artificial seawater was prepared by dissolving 100% natural sea salt in deionized water (26.7 g/L) to obtain a solution containing the same concentration of sodium as natural seawater (0.469 mol/kg).

### Example 4

### ELISA Assays

100 μL of TNB-ovalbumin complex at 10 mg/mL in 0.1M sodium bicarbonate were added to each well of a 96-well plate and plates incubated overnight at 4° C. After washing with 0.1% PBST, PBS 4% milk (200 μL/well) was added to block uncoated sites. TNT or TNT analogs at concentrations specified in each experiment and mouse anti-TNT antibody (0.5 μg/mL) were added to each well and plates incubated for ˜2 hours with gentle shaking. After washing with 0.1% PBST, HRP-conjugated goat anti-mouse antibody (100 ng/mL) was added to each well, plates incubated for 1 hour, and washed again. 100 μL/well of TMB substrate were added, and, after 10 minutes, 50 mL/well of 1M phosphoric acid were added to stop the reaction. The absorbance at 450 nm was measured with a GeneMate UniRead 800 plate reader. The cross reactivity (CR) of the anti-TNT antibody with each compound was evaluated as follows:

$$CR = \frac{{C}_{0}}{C} \times 100 \%$$

In the formula, C₀ is the concentration of TNT upon 50% of antibody displacement, and C is the concentration of compound used to achieve 50% displacement.

### Example 5

### Functionalization of the Crystal Surface

Crystals were washed with a mixture of hydrogen peroxide and ammonia hydroxide at 75° C. in a UV-ozone cleaner (novascan) under 5 psi oxygen. Dithiobis (succinimidyl propionate) (DSP) was used as a cross-linker to immobilize TNB-ovalbumin complex on the gold surface of crystals. Crystals were first immersed in DSP (1 mg/mL in DMSO) for 30 min, and then in TNB-ovalbumin complex (100 μg/mL) for 2 hour to form a “sandwich structure: Au [symbol] DSP [symbol] TNBovalbumin”, on the surface. Crystals were incubated in 1M Tris overnight to block uncoated sites.

### Example 6

### QCM Assays

The QCM system used in this study was a Q-sense E4 system (Q-Sense, Vastra Frolunda, Sweden), which measures changes in mass and related viscoelastic properties. The AT-cut QCM crystal used has a resonance frequency of 5 MHz. Using the Sauerbrey equation (Zeitschrift Fur Physik (1959)155: 206-222), 1 Hz frequency change can be converted to a mass change of 17.7 ng/cm2 on the crystal surface. The viscoelastic properties can be obtained from energy dissipation measured by the decay of oscillation. After immobilization of TNB-ovalbumin complex, anti-TNT antibody and solutions of TNT or TNT analogs were flowed sequentially at 50 μl/min at 25° C. The frequency change measured after addition of TNT was divided by the frequency change caused by the addition of antibody (Δf_{dis}). Δf_{dis} obtained from each compound tested was divided by the Δf_{dis} of the control sample (crystal without TNT or TNT analog), and the new parameter obtained, the normalized frequency change (Δf_{N}), was used for the data analysis.

$$Δ {f}_{dis} = \frac{Δ {f}_{TNT}}{Δ {f}_{Ab}} , Δ {f}_{N} = \frac{Δ {f}_{dis} of each compound}{Δ {f}_{dis} of control sample} \qquad ( 1 )$$

The ratio of frequency change over a chosen time interval, defined as “quick” slope k, was calculated as follows:

$$k = \frac{Δ {f}_{N}}{Δ t} \qquad ( 2 )$$

In the above formula, Δf_{N} is the normalized frequency change during the time interval Δt

### Example 7

### Mathematical Modeling of Antibody Displacement on QCM

The displacement of antibody on QCM can be simulated by assuming a Langmuir isotherm model. The deduction steps of the model have been previously reported. See Talanta. 2005. 68: 305-311; and Sens Actuators. 1997. B 42: 89-94. After the displacement step reaches equilibrium, the following equation describes the relationship between TNT concentration and the frequency change measured.

$$\frac{[ TNT ]}{Δ f} = \frac{[ TNT ]}{Δ {f}_{max}} + \frac{1}{Δ {f}_{max} {K}_{A}} \qquad ( 3 )$$

In the above formula, Δf is the frequency change at a given concentration of TNT, Δfₘₐₓ is the maximal frequency change when the antibody is completely displaced, and K_{A} is the binding affinity constant, as determined by the following formula:

$${K}_{A} = \frac{{k}_{A}}{{k}_{- A}} = \frac{θ}{( 1 - θ ) [ TNT ]} ⇒ θ = \frac{{K}_{A} [ TNT ]}{1 + {K}_{A} [ TNT ]} = \overline{Δ} {f}_{max} \qquad ( 4 )$$

In addition, Δfₘₐₓ and K_{A} were calculated from the plot of [TNT]/Δf with respect to [TNT]. In order to simulate the data obtained from QCM, the kinetics of antibody displacement was derived as described in Example 14. Briefly, the expression describing the formation of the antibody-TNT complex (Equation S-5 in Example 14) was integrated (Equation S-6 in Example 14), to calculate the forward reaction rate k_{A}. An ODE describing the change of Δf as a function of time was obtained from the expression of k_{A} and analyzed in Matlab. The model derived was used to analyze the experimental data, and predict the kinetics of displacement under different conditions, such as different pH and temperature.

$$\frac{d Δ f}{d t} = {k}_{A} [ TNT ] Δ {f}_{max} - ( {k}_{A} [ TNT ] + \frac{{k}_{A}}{{K}_{A}} ) Δ f \qquad ( 5 )$$

### Example 8

### Detection of TNT Through Antibody Displacement by ELISA

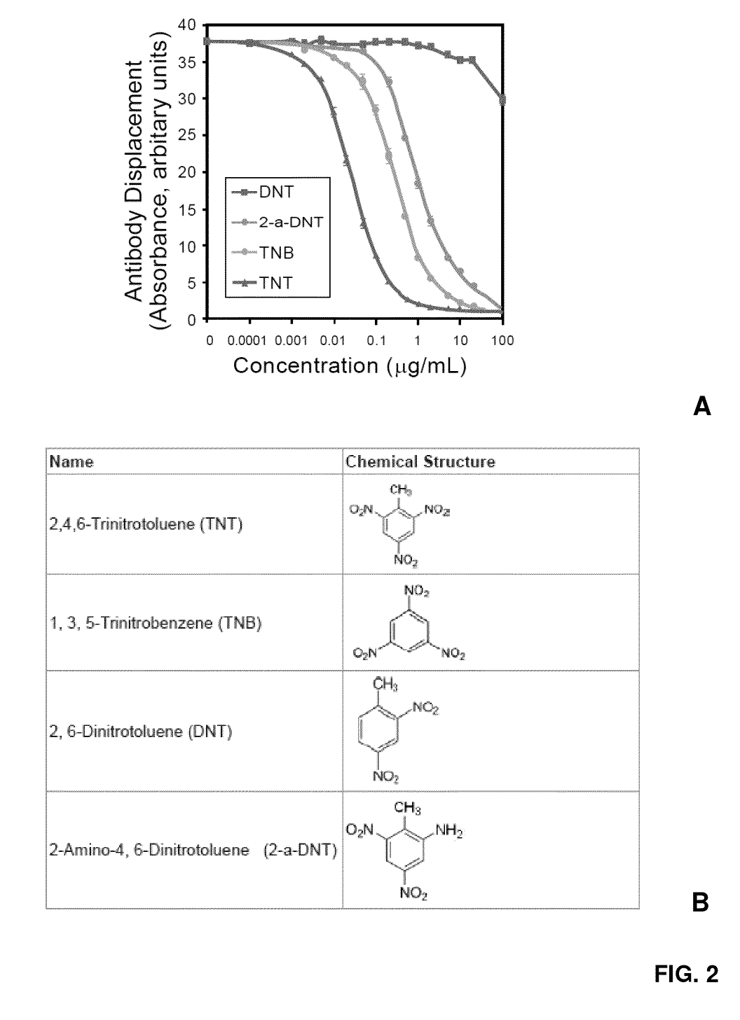

The anti-TNT monoclonal antibody A1.1.1 was chosen because of its high binding specificity to TNT compared to other nitro aromatic compounds with similar structures. ELISA analyses were first conducted to evaluate the feasibility of the displacement assay and determine the affinity and limit of detection of this antibody with a currently well-established technique. The principle of the displacement assay is based on the ability of the anti-TNT antibody to cross react with TNT analogs. TNB, which was previously reported to bind to this anti-TNT antibody with low affinity was used as a reference. The ability of TNT and other TNT analogs (including TNB) to displace the antibody from TNB was evaluated. Measurements of antibody displacement are reported in FIG. 2A using TNT, TNB, DNT, and 2-a-DNT. The structures of these molecules are shown in FIG. 2B. TNT was observed to cause the maximum antibody displacement, and the limit of detection was estimated to be 1 ng/mL.

Similar to most antibodies, the anti-TNT antibody displays cross-reactivity to compounds structurally similar to TNT. To quantify the binding affinity, the cross reactivity (CR) of the antibody for each compound was calculated as shown in Equation 1. Table 1 summarizes the cross-reactivity of these compounds to the anti-TNT antibody.

TABLE 1
Cross-reactivity for the compounds using antibody A1.1.1.

| | Concentration upon 50% of antibody | |
|---|---|---|
| Compound | displacement, μg/mL | CR, % |
| TNT | 0.025 | 100 |
| TNB | 0.3 | 8.3 |
| 2-a-DNT | 0.94 | 2.6 |
| DNT | >100 | <0.02 |

The results obtained illustrate the antibody relative affinity: TNT>TN>2-a-DNT>DNT. Furthermore, these results confirm previously reported measurements.

### Example 9

### Development of a QCM-Based Displacement Assay for TNT Detection

The method developed for QCM detection of TNT is based on the principle of antibody displacement described above. The TNB-ovalbumin complex was first immobilized onto the surface of the crystal. The surface was then saturated with anti-TNT antibody. Subsequently, a flux of TNT or TNT analogs was used to displace the antibody. The change in frequency was recorded until a plateau was reached, indicating maximum displacement of antibody. Because the molecular weight of the antibody is much greater than that of the TNT molecule, detecting the frequency change caused by the antibody displacement, rather than that associated with the binding of TNT, gives rise to significant amplification of the detection signal. This enables the sensor to achieve higher sensitivity and lower limit of detection.

Control studies were conducted to measure the frequency change caused by flowing TNT on a crystal coated with TNB-ovalbumin but without anti-TNT antibody, which resulted in signals indistinguishable from the background (e.g. the frequency change associated with flux of PBS buffer on to a TNB-ovalbumin coated crystal). The results indicated the absence of non-specific binding (data not shown).

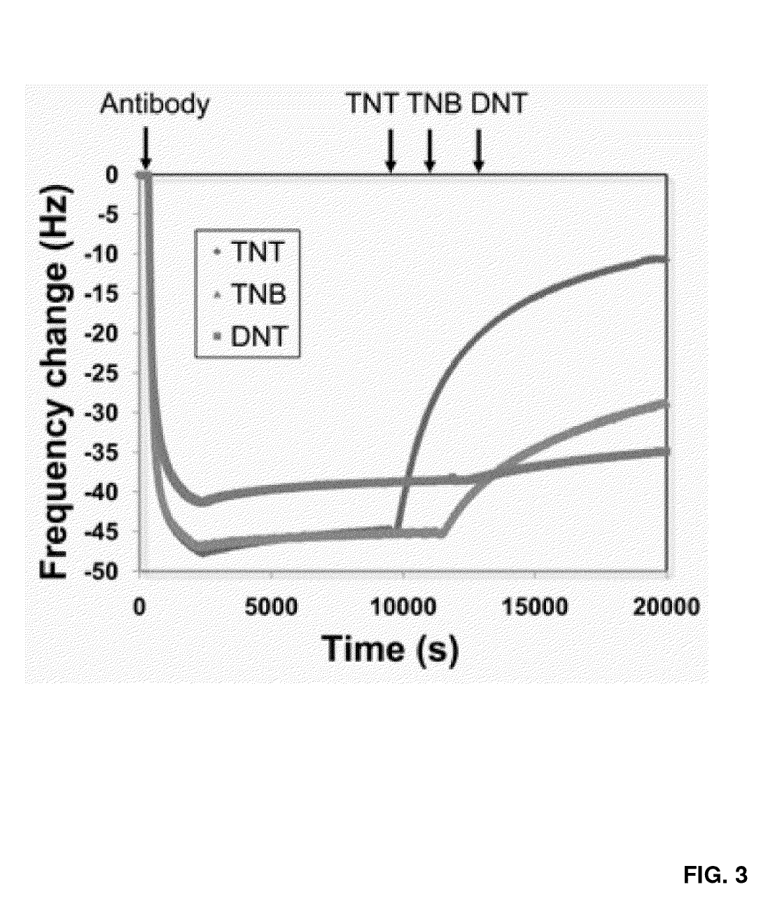

The antibody displacements by TNT and two analogous compounds, TNB and DNT, at a concentration of 10 μg/mL, were tested by QCM. See FIG. 3. A sharp decrease of frequency (around 45 Hz on average) is observed upon binding of the antibody to the TNB-ovalbumin complex on the crystal surface, which corresponds to a mass change of 506.25 ng from the Sauerbrey equation. The number of antibody molecules attached on sensor surface is 2.03×10¹². Given the size of crystal (9 mm diameter) and ovalbumin molecule (6.1 nm diameter), Applicants estimated that 2.18×10¹² molecules of ovalbumin are immobilized on the sensor surface. Hence, Applicants can assume that virtually all TNB-ovalbumin molecules form a complex with the antibody. After immobilization of the antibody, the solution of compound was flowed on to the crystal and the displacement of antibody was monitored. See FIG. 3.

To minimize the variability of surface immobilization, (frequency change of TNT/frequency change of antibody) was used in the data analysis (Equation 2). The displacement caused by TNT was about 3-fold higher than that observed using DNT. The extent of displacement (the magnitude of frequency change) obtained at equilibrium reflects the binding affinity of each compound for the anti-TNT antibody. The antibody displacement measured by QCM is in agreement with the relative affinity of the three compounds calculated by ELISA: TNT>TNB>DNT.

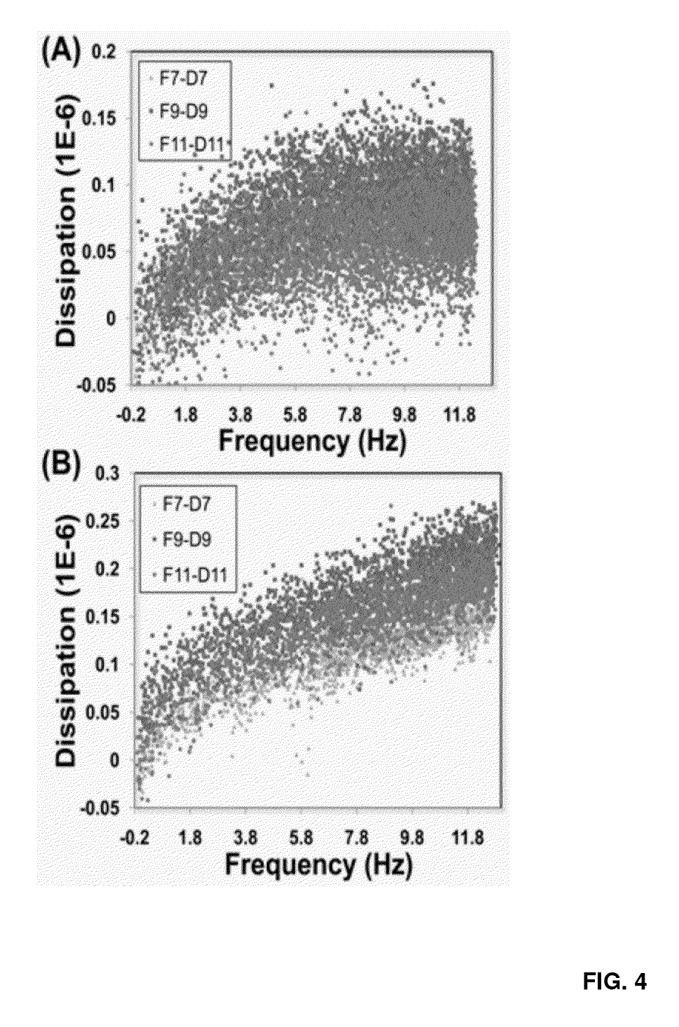

The results presented in this Example suggest that this sensor allows distinguishing TNT from molecules with similar chemical structure when they are present in solution at similar concentrations. However, the frequency change associated with a solution containing low TNT concentration is expected to be similar to that of a solution containing high TNB concentration. To address this issue, Applicants considered the energy dissipation. The change of energy dissipation, ΔD, was related to the change in frequency, ΔF, thereby removing the time dependency of the data. The slope in ΔD versus ΔF plot indicates different states (conformations) of proteins (anti-TNT antibody in this Example). Results from QCM revealed that 1 μg/mL TNT (Δf=26.65 Hz) induces a frequency change similar to 10 μg/mL TNB (Δf=28.85 Hz). However, the ΔD/Δf ratio of 1 μg/mL TNT (ΔD/Δf=0.0051±0.0011×10⁻⁶/Hz) was significantly lower than that of TNB (ΔD/Δf 0.0111±0.0007×10⁻⁶/Hz), as easily appreciated by comparing the slopes of the ΔD versus Δf plot reported in FIG. 4, which indicates that TNT can be distinguished from TNB, even when they induce similar signals on QCM. In sum, Applicants demonstrated in this Example that QCM can be used to effectively distinguish TNT from other molecules with similar chemical structure.

### Example 10

### Limit of Detection of the QCM Based TNT Sensor

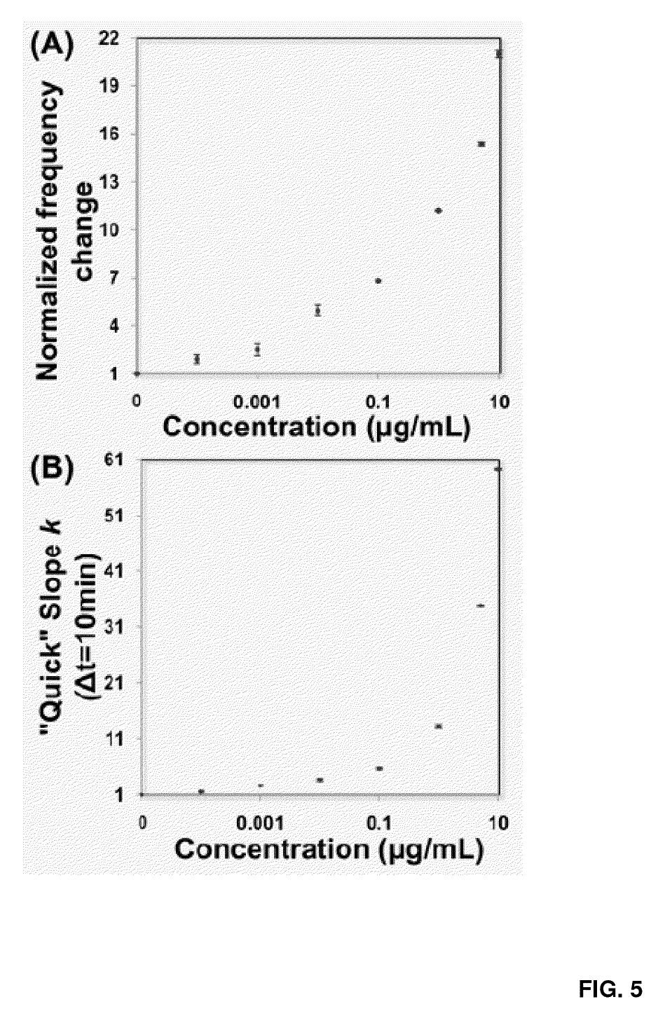

The QCM based sensor described here allows detecting TNT with high specificity in solution, and distinguishing it from molecules with similar chemical structure. Applicants investigated the detection limit (sensitivity) of this sensor by testing solutions of TNT ranging from 0.1 ng/mL to 10 μg/mL. The extent of antibody displacement was proportional to the concentration of TNT in solution. The data analysis was based on the use of the normalized frequency change Δf_{N} (Equation 2), which was calculated by dividing the Δf_{dis} of each compound by Δf_{dis} of a control sample without TNT. Thus, the resulting values are generally equal or greater than one. The normalized frequency changes of TNT at different concentrations are reported in FIG. 5A. The lowest detectable concentration of TNT in this assay was 0.1 ng/ml(p<0.01), which is one order of magnitude lower than what was previously determined by ELISA (1 ng/mL), demonstrating the optimal properties of a QCM based sensor for applications in the field.

### Example 11

### Accelerated Detection Using the Rate of Antibody Displacement

The analysis described above is based on the measurements of the frequency change at equilibrium caused by the displacement of anti-TNT antibody after the addition of TNT, which usually requires several hours. This time scale is typically not considered practical for rapid on-site detection. Hence, Applicants introduced a new parameter, the “quick” slope k (Equation 3), which allows estimating the dependence of the rate of displacement on the concentration of TNT.

The “quick” slope k was calculated for solutions of TNT ranging from 0.1 ng/mL to 10 μg/mL, and a time scale of 10 minutes. See FIG. 5B. The “quick” slope was observed to increase with the concentration of TNT, even at low TNT concentrations (0.1 ng/mL, p<0.01). Therefore, the “quick” slope k can be used to reliably quantify the detection of TNT in solutions of different TNT concentrations (0.1 ng/mL-10 μg/mL) while considerably decreasing the time of detection (˜10 minutes).

### Example 12

### Detection of TNT in Crude Environments

The experiments described above were conducted using solutions of TNT in PBS buffer, which may not reflect the conditions of on-site analysis. Thus, in an attempt to evaluate the reliability and robustness of this sensor for use in the field, solutions of TNT crowded with molecules with similar chemical structure, which might interfere with the detection, were tested. Particularly, Applicants used a solution of commercially available fertilizer, which contains nitrogenous compounds, with chemical reactivity potentially similar to TNT and TNT analogs. Applicants also used seawater, which represents a commonly contaminated environment.

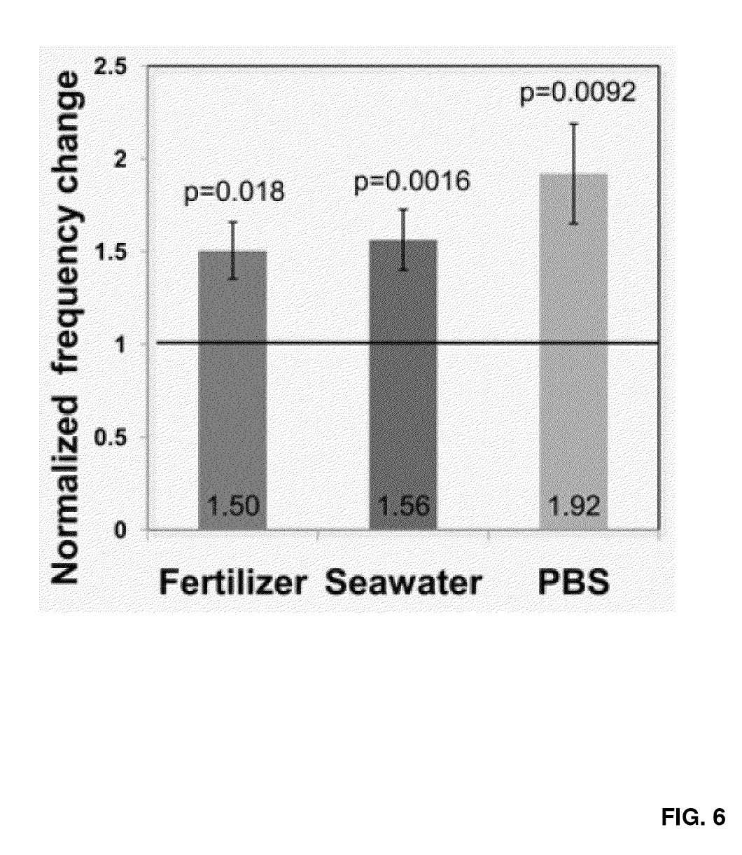

The rate of displacement and normalized frequency change at equilibrium for solutions of TNT in PBS buffer, in fertilizer, and in artificial seawater were found to be comparable (data not shown), demonstrating the robustness of this detection method. Next, Applicants investigated the limit of detection of TNT in crude environments and compared it to PBS (0.1 ng/mL). See FIG. 6. The normalized frequency changes obtained from the fertilizer solution and seawater were 1.50±0.15 and 1.56±0.16, respectively (p<0.05), which are comparable to the normalized frequency change measured using PBS (1.92±0.27, p<0.01), indicating that the QCM based detection of TNT is not limited by the composition of the solution.

### Example 13

### Mathematical Modeling and Calculation of Binding Affinity Constants

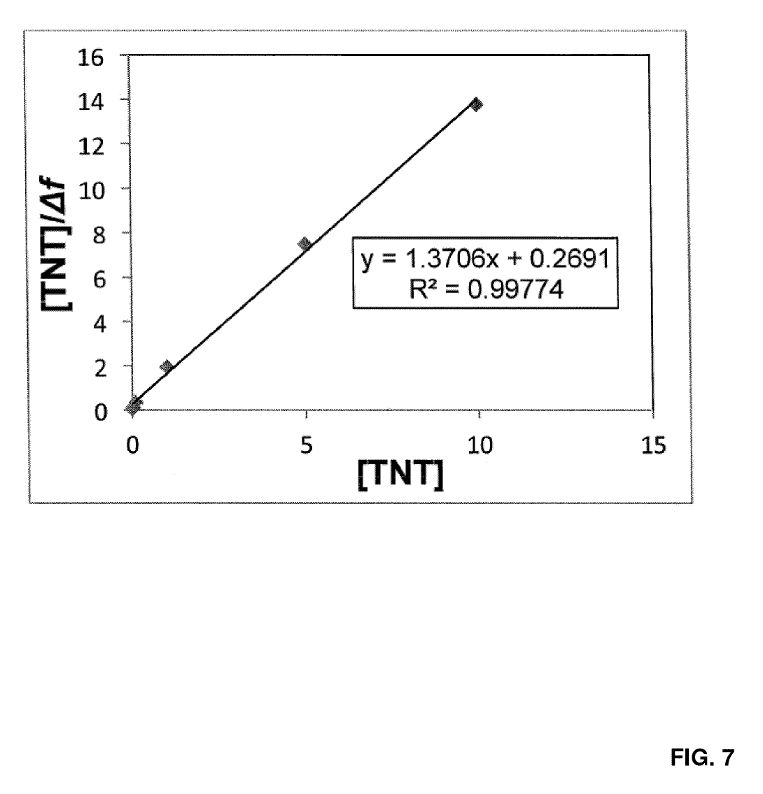

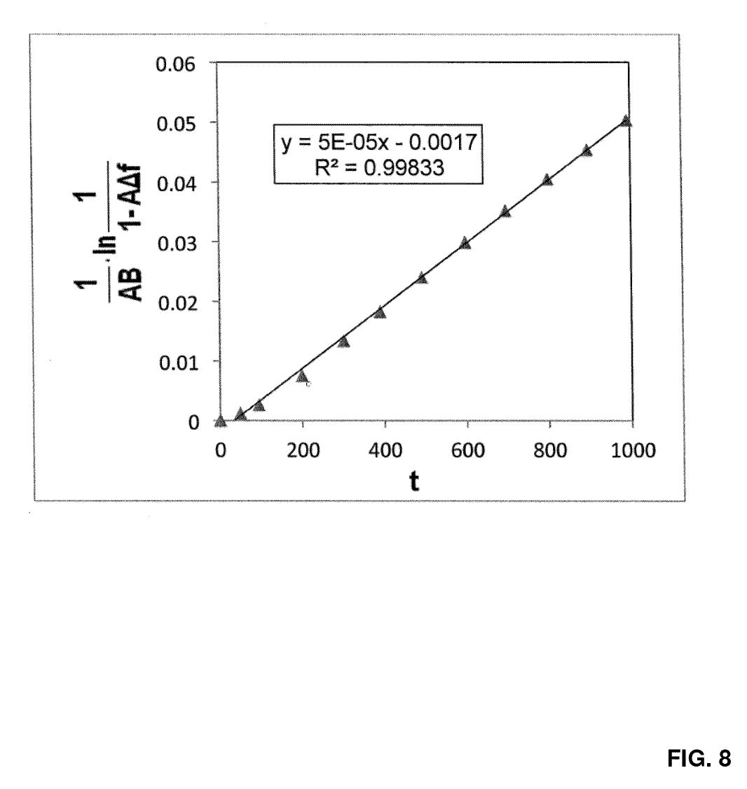

The displacement process can be modeled with a Langmuir isotherm model, which was previously used to describe antigen-antibody interactions on a surface. The binding affinity constant K_{A} refers to the relative affinity of the anti-TNT antibody to a specific compound, and was determined based on Equation 4. See FIG. 7. The maximum frequency change Δfₘₐₓ caused by antibody displacement and TNT binding affinity constant K_{A} are 0.73±0.05 and 5.09±0.61, respectively. The positive reaction rate k_{A} (Equation S-5 in Example 14 and FIG. 8) was (5.247±0.027)×10⁻⁵. The data obtained with solutions of TNB and DNT were analyzed in the same fashion and results are reported in Table 2.

TABLE 2
Parameters obtained from the mathematical model.

| Parameter | Δfₘₐₓ | K_{A} (mL/μg) |
|---|---|---|
| TNT | 0.73 ± 0.05 | 5.09 ± 0.61 |
| TNB | 0.66 ± 0.04 | 1.44 ± 0.62 |
| DNT | 0.24 ± 0.11 | 0.47 ± 0.14 |

The values of both Δfₘₐₓ and K_{A} represent the relative affinity of each molecule for the anti-TNT antibody, with the largest value corresponding to the highest affinity. It is important to notice that the relative affinity values calculated in this study refer to the ability of each molecule to displace anti-TNT antibody specifically from TNB. Thus, as shown in Table 2, the relative affinity values (TNT>TNB>DNT) are consistent with the experimental results obtained.

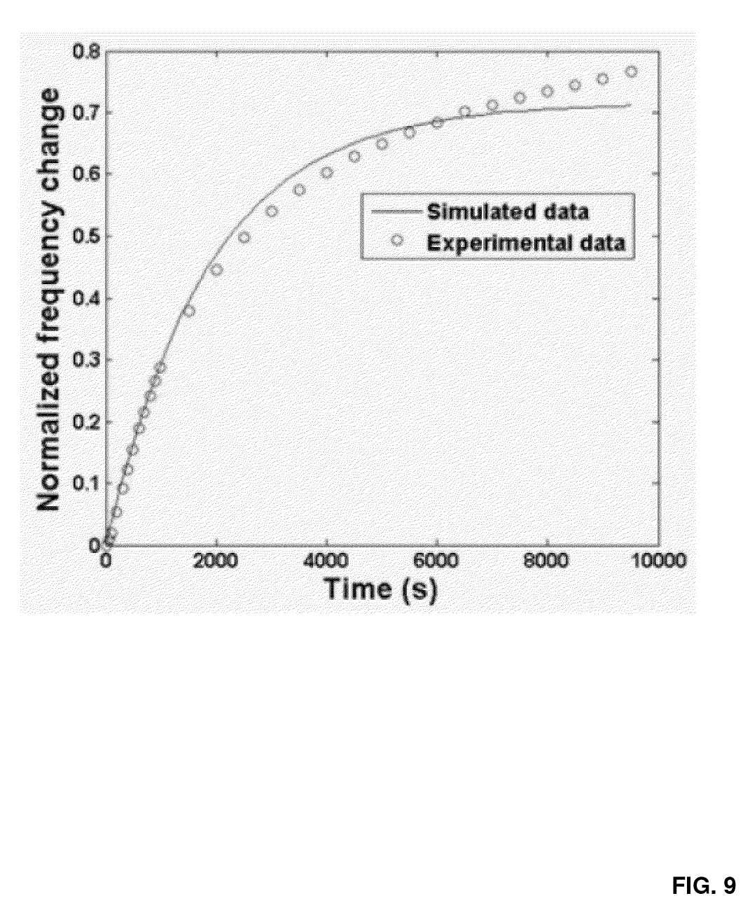

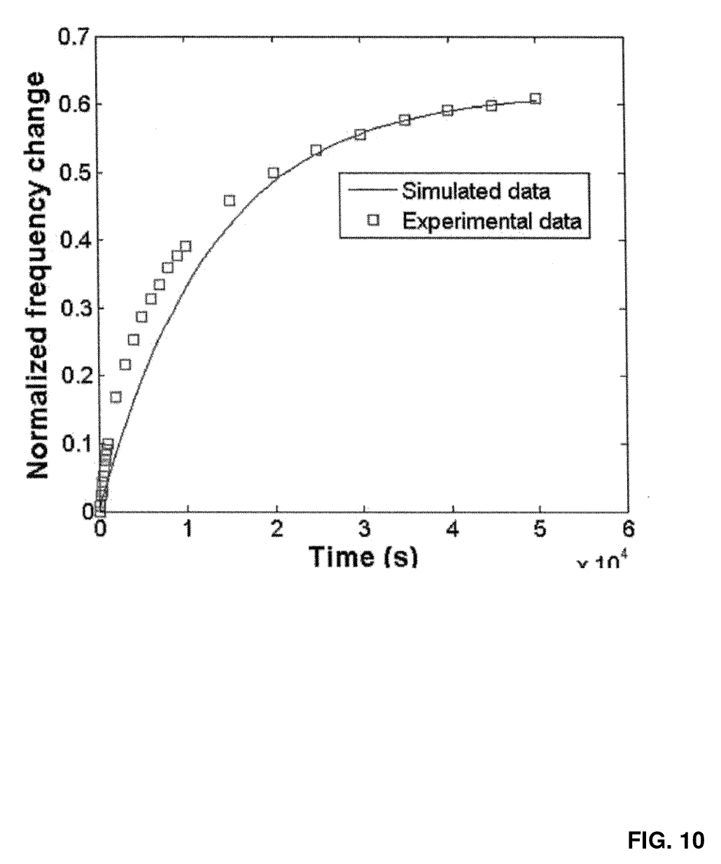

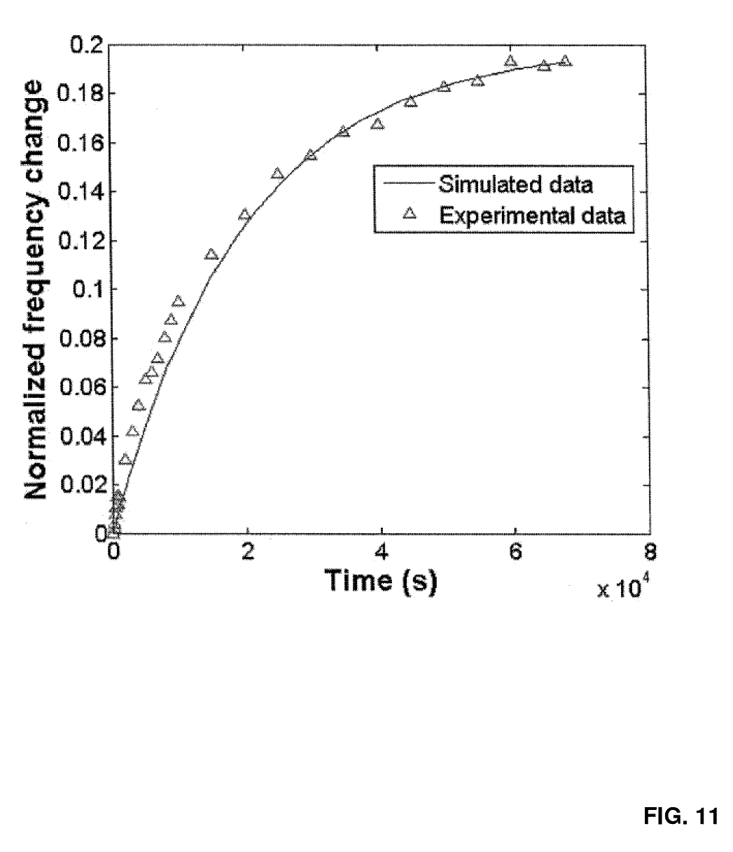

With known values for Δfₘₐₓ, K_{A} and k_{A}, the ODE describing the change of Δf as a function of time (Equation 5) can be analyzed in Matlab. See FIG. 9. The detection of TNT in a solution at 10 μg/mL was simulated and observed to fit the experimental data accurately during the initial phase of displacement. Similar results were obtained for simulated and experimental data using lower TNT concentration (1 μg/mL, data not shown). The model was therefore considered acceptable and extended to the analysis of TNB and DNT at high concentrations. See FIGS. 10-11. The mathematical model developed allows establishing a standard curve, which can be used to estimate the concentration of unknown TNT solutions.

### Example 14

### Deduction Steps to Construct the Model

There are two main steps in the aforementioned displacement assays: attachment step and displacement step. Displacement takes place between TNT and TNB-ovalbumin towards anti-TNT antibody. The process includes two immunoreactions:

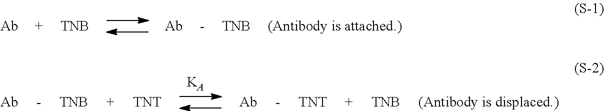

“Ab” above represents anti-TNT antibody. “K_{A}” is the binding affinity constant. The affinity constant plays an important role in the adsorption process, and can be used to study interaction between two molecules. Thus, it is envisioned that K_{A} is a significant factor in improving detection performance.

In the process above, the first step (attachment) is ignored because the crystal surface is saturated by antibody. Thus, only the second step (displacement) is considered. The following assumptions are used in the model: the crystal surface is saturated by anti-TNT antibody before the addition of TNT; the antibody binds one molecule of TNT or TNB so that if one of them is bound to antibody, the others will be free; the displacement of antibody caused by TNT reaches equilibrium after a certain time; and the concentration of TNB is constant on surface. The displacement step can reach equilibrium after a certain time, which satisfies following equation:

k_{A}[TNT](1−θ)=k_{-A}[TNB]θ [symbol] k_{A}[TNT](1−θ)=k_{-A}θ (S-3)

In this equation, 0 is the fraction of the surface without coverage of antibody. On the contrary, (1−0) represents the sites that are available for TNT. The effect of TNB concentration is ignored because it is immobilized on surface. By defining K_{A} as the binding affinity constant, one can obtain the expression for 0. Thereafter, one can obtain an equation with a relation between TNT concentration and frequency change as follows:

$$\begin{matrix}{K}_{A} = \frac{{k}_{d}}{{k}_{- A}} = \frac{θ}{( 1 - θ ) [ TNT ]} ⇒ θ = \frac{{K}_{A} [ TNT ]}{1 + {K}_{A} [ TNT ]} = \frac{Δ f}{Δ {f}_{max}} & ( S \text{-} 4 ) \\ \begin{matrix}⇒ \frac{1}{Δ f} = \frac{1}{Δ {f}_{max}} + \frac{1}{Δ {f}_{max} {K}_{A} [ TNT ]} \\ ⇒ \frac{[ TNT ]}{Δ f} = \frac{[ TNT ]}{Δ {f}_{max}} + \frac{1}{Δ {f}_{max} {K}_{A}}\end{matrix} & ( 4 )\end{matrix}$$

In Equation 4, [TNT] is the molar concentration of TNT. Δf is the frequency change caused by a given concentration of TNT. Δfₘₐₓ is the maximal frequency change when all the antibodies are displaced. Since [TNT]/Δf is linear to [TNT], Δfₘₐₓ and K_{A} can be obtained from the slope and intercept of the plot of [TNT]/Δf with respect to [TNT].

The equations above indicate displacement of antibody from a solid surface. Since the data from QCM assays varied as a function of time, these equations cannot be used to simulate the data directly. To evaluate the kinetics of the displacement process, Applicants have taken into account the rate of adsorption. In Equation S-2, the expression of the forward reaction rate (rate of formation of Ab-TNT) is given by:

$${r}_{Ab \text{-} TNT} = {k}_{A} [ TNT ] ( 1 - θ ) - {k}_{- A} θ = {k}_{A} [ TNT ] - ( {k}_{A} [ TNT ] + {k}_{- A} ) θ \\ {r}_{Ab \text{-} TNT} = - \frac{d [ TNT ]}{d t} = \frac{d θ}{d t} = {k}_{A} [ TNT ] - ( {k}_{A} [ TNT ] + {k}_{- A} ) θ \qquad ( S \text{-} 5 )$$

An ODE is then obtained. The concentration of TNT is presumably constant because TNT in solution is flowed continuously into the reaction chamber. As a result, the aforementioned equation can be integrated with respect to 0, from 0 time t. The result can be simplified with constant A and B (Equation S-5 in Example 14). With the values of Δfₘₐₓ and K_{A}, A and B can be calculated. In this case, only lower K_{A} (i.e., the positive reaction rate) is unknown. Applicants can plot the right part of the equation as a function of time. The new plot provides the value of lower K_{A}. Lower K_{A} can then be obtained.

$$⇒ t = \frac{1}{{k}_{A} [ TNT ] + {k}_{- A}} ln \frac{{k}_{A} [ TNT ]}{{k}_{A} [ TNT ] - ( {k}_{A} [ TNT ] + {k}_{- A} ) θ} \\ Q θ = \frac{Δ f}{Δ {f}_{max}} \\ ∴ {k}_{A} t = \frac{1}{AB} ln \frac{1}{1 - A Δ f} \qquad ( S \text{-} 6 )$$

In the aforementioned equation, A=(1+(1/K_{A}[TNT]))/Δfₘₐₓ, and B=[TNT]. With the equations above, a new ODE with respect to Δf can be obtained, as shown in Equation 5. With the known values of Δfₘₐₓ, K_{A} and k_{A}, this ODE can be analyzed at a certain TNT concentration using Matlab. The frequency change caused by TNT as a function of time can be simulated. This can be compared with the aforementioned experimental data and used to predict the kinetic behavior under unknown conditions.

$$\frac{d Δ f}{d t} = {k}_{A} [ TNT ] Δ {f}_{max} - ( {k}_{A} [ TNT ] + \frac{{k}_{A}}{{K}_{A}} ) Δ f \qquad ( 5 )$$

### Summary of Examples 1-14

In sum, Applicants have developed a rapid and accurate QCM based displacement assay for the detection of TNT in liquid phase by exploiting the cross reactivity of an anti-TNT antibody (A1.1.1) for TNT analogs. In the aforementioned Examples, ELISA was first used to evaluate the displacement assay for the detection of TNT. The relative affinity of the antibody for TNT and selected TNT analogs, and the limit of detection of TNT (1 ng/mL) were then calculated. Results obtained were comparable to previously reported data. The displacement principle was subsequently used to develop a QCM based assay for the detection of TNT with higher sensitivity and specificity. The limit of detection obtained was an order of magnitude lower than previously reported (0.1 ng/mL). The robustness of this QCM based TNT sensor was confirmed by evaluating TNT detection in crude environments, such as fertilizer and seawater. Furthermore, the limit of detection achieved was comparable to that measured in pure TNT solutions.

The aforementioned Examples are based on the competition between two immunochemical interactions: the binding TNB-antibody and the binding TNT-antibody. Therefore, the extent of antibody displacement depends on the ratio between the relative binding affinities of TNT and TNB for the antibody. The limit of detection could be further increased by using a TNT analog with lower affinity than TNB as surface competitors. On the other hand, using a competitor with high affinity is likely to increase the specificity of the assay for detection of TNT in crude environments. The use of protein engineering technologies for the selection of an anti-TNT antibody with enhanced affinity for TNT or lowered cross-reactivity with TNT analogs would also allow further enhancing the sensitivity and specificity of this detection method.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of detecting a molecule in a sample, wherein the method comprises:
associating the sample with a complex, wherein the complex comprises:
an antigen, and
a binding agent associated with the antigen;
measuring a rate of displacement of the binding agent from the antigen by the molecule in the sample,
wherein the measuring step comprises a determination of a change in frequency of the sample and a change in energy dissipation of the sample over a time interval; and
correlating the measured rate of displacement to presence of the molecule in the sample,
wherein the correlating step comprises a calculation of a ratio of a change in energy dissipation of the sample over the change in frequency of the sample over the time interval.

2. The method of claim 1, wherein the correlating step comprises calculating the slope of a plot, wherein the plot reflects the change in energy dissipation of the sample over the change in frequency of the sample over the time interval.

3. The method of claim 1, wherein the method comprises the utilization of at least one of quartz crystal microbalance (QCM), surface plasmon resonance (SPR), ellipsometry, microcantilevers, optical microcavities, a Langmuir kinetic model, and combinations thereof.

4. The method of claim 1, wherein the method comprises the utilization of quartz crystal microbalance (QCM).

5. The method of claim 1, wherein the sample is in a liquid state.

6. The method of claim 1, wherein the sample is in a gaseous state.

7. The method of claim 1, wherein the sample is derived from a gaseous environment.

8. The method of claim 1, wherein the sample is derived from a liquid environment.

9. The method of claim 1, wherein the molecule comprises an explosive.

10. The method of claim 9, wherein the explosive is selected from the group consisting of nitroglycerin, 2,4,6-trinitrotoluene (TNT), pentaerythritol tetranitrate (PETN), octahydro-1,3,5,7-tetranitro-1,3,5,7-tetrazocine (HMX), nitrocellulose, analogs thereof, derivatives thereof, and combinations thereof.

11. The method of claim 1, wherein the molecule is selected from the group consisting of 2,4,6-trinitrotoluene (TNT), analogs thereof, derivatives thereof, and combinations thereof.

12. The method of claim 1, wherein the molecule is 2,4,6-trinitrotoluene (TNT).

13. The method of claim 11, wherein the antigen is selected from the group consisting of 2,4,6-trinitrotoluene (TNT), analogs thereof, derivatives thereof, and combinations thereof.

14. The method of claim 11, wherein the antigen is selected from the group consisting of 1,3,5-Trinitrobenzene (TNB), 2,6-Dinitrotoluene (DNT), 2-Amino-4,6-Dinitrotoluene (2-a-DNT), 2,4,6-trinitrobenzene (TNB), 2,4,6-trinitrobenzene sulfonic acid (TNBS), derivatives thereof, and combinations thereof.

15. The method of claim 1, wherein the binding agent has an affinity for the antigen, and an affinity for the molecule.

16. The method of claim 1, wherein the binding agent is selected from the group consisting of aptamers, peptides, peptide nucleic acids, antibodies, proteins, RNA, DNA, small molecules, dendrimers, and combinations thereof.

17. The method of claim 11, wherein the binding agent is an anti-TNT antibody.

18. The method of claim 17, wherein the anti-TNT antibody is a monoclonal antibody.

19. The method of claim 1, wherein the binding agent is unlabeled.

20. The method of claim 1, wherein the method takes from about 30 seconds to about 60 minutes.

21. The method of claim 1, wherein the method determines the concentration of the molecule in the sample.

22. The method of claim 1, wherein the method is capable of detecting nanogram levels of the molecule in the sample.
